(19) United States
(12) Patent Application Publication
(10) Pub. No.: US 2026/0081082 A1
(43) Pub. Date: Mar. 19, 2026

(54) SPIRO-BASED IONIC LIQUID ELECTROLYTE FOR LOW TEMPERATURE SUPERCAPACITORS AND METHODS OF FABRICATING SAME

(71) Applicant: 10644137 CANADA INC., Calgary (CA)

(72) Inventors: Damilola Yusuf Momodu, Calgary (CA); Hamid Pahlevaninezhad, Lexington, MA (US); Majid Pahlevaninezhad, Kingston (CA); Sam Scherwitz, Calgary (CA)

(73) Assignee: 10644137 CANADA INC., Calgary, AB (CA)

(21) Appl. No.: 19/109,983

(22) PCT Filed: Sep. 7, 2023
(86) PCT No.: PCT/CA2023/051182

§ 371 Date: Mar. 7, 2025

Related U.S. Application Data
(60) Provisional application No. 63/404,409, filed on Sep. 7, 2022.

(51) Int. Cl. H01G 11/62; H01G 11/60

(52) U.S. Cl. CPC H01G 11/62; H01G 11/60

(57) ABSTRACT

A method for fabricating an ionic liquid electrolyte such as a spiro-based ionic liquid electrolyte, the method has the steps of: synthesizing an intermediate spiro-based product, and applying an ionic exchange process to the intermediate spiro-based product to obtain the ionic liquid electrolyte. The obtained an ionic liquid electrolyte comprising an ionic liquid salt with a cation of: (I) The ionic liquid electrolyte may be used in an electrochemical energy-storage apparatus such as a supercapacitor.

### CROSS-REFERENCE TO RELATED APPLICATIONS

This application claims the benefit of U.S. Provisional Patent Application Ser. No. 63/404,409, filed Sep. 7, 2022, the content of which is incorporated herein by reference in its entirety.

### FIELD OF THE DISCLOSURE

The present disclosure relates generally to supercapacitor, and in particular to spiro-based ionic liquid electrolyte thereof and methods of fabricating same.

### BACKGROUND

The current interest in reducing global emissions affecting climate change as well as migrating to a greener and sustainable energy generation lifestyle has led to the skyrocketing demand in employing reliable and safe energy-storage technologies. Supercapacitors have attracted a tremendous amount of interest due to their surface-related electrochemistry which, compared to batteries, enables rapid charge-discharge rate, long cycle life, low maintenance, less restrictive operating temperature range, and safety. These properties make supercapacitors ideal for direct incorporation into renewable energy generation systems (such as solar technologies), where there is a significant change in ambient temperature during the daytime and different seasons of the year. However, the low energy density of supercapacitors limits their ability to store energy when compared to batteries. Since the electrolyte plays a major role on both energy density and temperature limitations, it is important that supercapacitors may have electrolytes with enhanced chemical and physical properties for mitigating problems that may otherwise degrade their performance.

### SUMMARY

Embodiments disclosed herein relate to electrochemical energy-storage devices and methods of fabricating same. In some embodiments, the electrochemical energy-storage devices are high energy-volume capacitors (also called “supercapacitors”) for storing therein electrical energy that may be used as a power source.

According to one aspect of this disclosure, there is provided a supercapacitor comprising a cathode layer, an anode layer, and a separator layer between the cathode and anode layers. The separator layer comprises an ionic liquid electrolyte.

According to one aspect of this disclosure, there is provided an electrochemical energy-storage apparatus comprising: an ionic liquid electrolyte comprising an ionic liquid salt with a cation of:

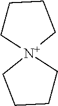

In some embodiments, the ionic liquid electrolyte is a spiro-based ionic liquid electrolyte.

In some embodiments, the ionic liquid electrolyte is spiro-1,1′ bipyrrolidinium bromide (SBPBr):

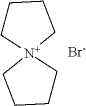

According to one aspect of this disclosure, there is provided an electrochemical energy-storage apparatus comprising: an ionic liquid electrolyte comprising an ionic liquid salt with an anion of:

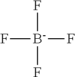

In some embodiments, the ionic liquid electrolyte is a spiro-based ionic liquid electrolyte.

In some embodiments, the ionic liquid electrolyte is spiro-1,1′ bipyrrolidinium tetraflouroborate (SBPBF₄):

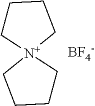

According to one aspect of this disclosure, there is provided a method for fabricating an ionic liquid electrolyte, the method comprising: synthesizing an intermediate spiro-based product; and applying an ionic exchange process to the intermediate spiro-based product to obtain the ionic liquid electrolyte.

In some embodiments, said synthesizing the intermediate spiro-based product comprises: adding a plurality of precursors are added to an organic solvent (such as isopropanol or acetonitrile) to obtain a mixture, and stirring the mixture for a period of time at a temperature of 340 Kelvin (K) to obtain the intermediate spiro-based product; and purifying the intermediate spiro-based product; the plurality of precursors comprise alkylation of a cyclic amine and a dihaloalkane.

In some embodiments, the organic solvent comprises isopropanol or acetonitrile.

In some embodiments, the period of time is about 6 hours (h), 12 h, 18 h, or 24 h.

In some embodiments, said purifying the intermediate spiro-based product comprises: washing the intermediate spiro-based product with acetone.

In some embodiments, said applying the ionic exchange process to the intermediate spiro-based product to obtain the ionic liquid electrolyte comprises: reacting the intermediate spiro-based product with hydrofluoroboric acid or an alkali-metal tetrafluoroborate salt in ethanol.

### BRIEF DESCRIPTION OF THE DRAWINGS

For a more complete understanding of the disclosure, reference is made to the following description and accompanying drawings, in which:

FIG. 1A is a schematic perspective view of a supercapacitor;

FIG. 1B is a schematic exploded view of the supercapacitor shown in FIG. 1A;

FIG. 2 is a plot showing the self-discharge profile of the supercapacitor shown in FIG. 1A for illustrating its low-temperature voltage leakage; and

FIG. 3 is a plot showing the voltage holding test profile of the supercapacitor shown in FIG. 1A for illustrating the capacitance retention thereof over time.

### DETAILED DESCRIPTION

Subsection F of the Detailed Description lists the references cited in this disclosure. The content of each of these references is incorporated herein by reference in its entirety.

### A. SUPERCAPACITORS AND SPIRO-BASED IONIC LIQUID ELECTROLYTE

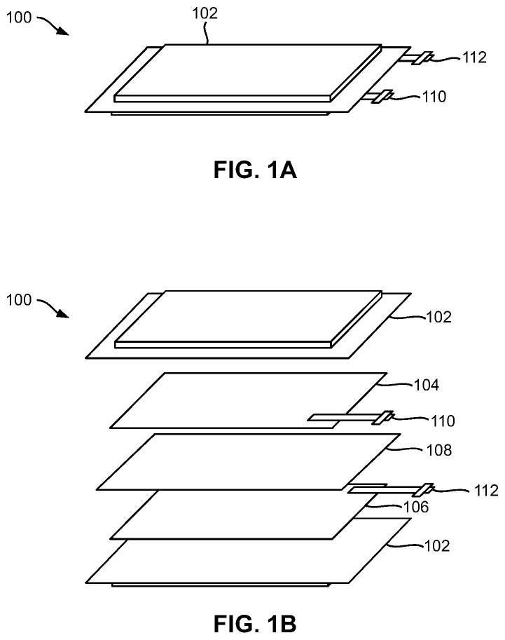

Turning now to FIGS. 1A and 1B, an electrochemical energy-storage devices in the form of a high energy-volume capacitor (also called “supercapacitor”) is shown and is generally identified using reference numeral 100. In these embodiments, the supercapacitors are highly stable, low temperature supercapacitors which may be operated at much lower temperatures compared to the operation temperatures of most prior-art supercapacitors.

As shown in FIGS. 1A and 1B, the supercapacitor 100 comprises a pair of cell casing 102 enclosing therein a cathode layer 104, an anode layer 106, and a separator layer 108 sandwiched between the cathode and anode layers 104 and 106. A cathode tab or electrode 110 and an anode tab or electrode are electrically connected to the cathode and anode layers 104 and 106, respectively, for electrically connecting the supercapacitor 100 to various electrical components or devices (not shown). For example, the cathode and anode electrodes 110 and 112 may be electrically connected to a power source such as a solar panel for storing electrical energy received from the solar panel. The cathode and anode electrodes 110 and 112 may be electrically connected to a power-consumption device for acting as a power source therefor and powering the power-consumption device.

In some embodiments, the separator layer 108 comprises an ionic liquid electrolyte which comprises an ionic liquid salt with a cation of:

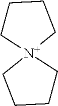

In some embodiments, the ionic liquid electrolyte comprises an ionic liquid salt with an anion of:

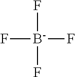

In some embodiments, the ionic liquid electrolyte is a spiro-based ionic liquid electrolyte, wherein the electrolyte with specific-sized assortment of ions serves as electronic charge transport and storage agents within the carbon pores of the supercapacitor electrodes. In some embodiments, the spiro-based ionic liquid electrolyte is in the form of a spiro-based ionic liquid salt, which is highly soluble in organic solvents.

Herein, the term “ionic liquid salt” refers to the solid white precipitate which is then dissolved in a suitable solvent to obtain the liquid form referred to as “ionic liquid electrolyte”. In various embodiments, the solvent may be any organic solvent such as acetonitrile (AN), polypropylene carbonate (PC), or a combination of organic solvents in varying volume proportions.

The nature of the ion dynamics and solvent adopted in the spiro-based ionic liquid electrolyte disclosed herein enables the supercapacitor to operate at low temperatures. For example, the operation of the ionic liquid electrolyte in a carbon-electrode supercapacitor provides energy storage at a wide temperature range such as from 60° C. to −60° C., while most prior-art commercial supercapacitors are only rated up to −45° C. With such a wide operating temperature range, the ionic liquid electrolyte disclosed herein provides various benefits to energy-storage devices such as hybrid supercapacitors (which contains both supercapacitor-type and battery-type active electrode materials), electric double-layer capacitors with carbon active electrode materials, Lithium-ion capacitors, and the like. Such devices have already been used as energy-storage components in numerous applications such as electric vehicles, outdoor lighting and display, smart devices, and the like.

In some embodiments, the spiro-based ionic liquid salt disclosed herein may be combined with polymer gels for the production of solid-state freestanding supercapacitors which may be integrated into other device structures such as the next generation flexible quantum-dot light-emitting diode (QLED) panels and QLED passive-matrix displays.

### B. FABRICATION METHOD OF THE SPIRO-BASED IONIC LIQUID ELECTROLYTE

In some embodiments, a bi-step process or method is used for fabricating the spiro-based ionic liquid electrolyte.

### Step I: Synthesis of Intermediate Spiro-Based Product

At this step, a plurality of precursors including the alkylation of a cyclic amine and a dihaloalkane are added to an organic solvent (such as isopropanol or acetonitrile), and the mixture is stirred for a period of time such as 6 hours (h), 12 h, 18 h, or 24 h at a temperature of 340 Kelvin (K) to allow a synthesis reaction thereof and produce a spiro-quaternary ammonium based intermediate (such as spiro ammonium halide). Then, purification is carried out by thoroughly washing the produced spiro-quaternary ammonium based intermediate with acetone to obtain a pure intermediate product (that is, spiro quaternary ammonium halide). The purification ensures that a pure intermediate compound is used for the ion exchange in creating the final product.

This step gives rise to a high yield of the intermediate of about 96%.

### Step II: Ionic Exchange Process to Final Ionic Liquid Salt

At this step, the halide intermediate obtained in step I is further reacted with hydrofluoroboric acid or an alkali-metal tetrafluoroborate salt in ethanol to produce the spiro quaternary ammonium salt.

In some embodiments, the halide intermediate may be treated in a basic medium to form a spiro ammonium hydroxide solution (for easy reaction with tetrafluoroborate anion precursors). Then, the spiro ammonium hydroxide solution is mixed with the hydrofluoroboric acid in ethanol in room temperature and is stirred for 18 h. After reaction, filtration is used to remove the precipitate. The spiro quaternary ammonium salt (that is, the spiro-based ionic liquid salt) is then obtained.

With the bi-step process, an extra purification step may not be required before integrating the spiro-based tetrafluoroborate ionic salt into an activated carbon supercapacitor. More specifically, the synthesis with hydrofluoroboric acid in basic media reduces the need of an extra purification step with repeated evaporation (see References [1] and [2]) to remove the halide-based by-product (which is impurity).

Thus, the bi-step process is a simple, economical, and scalable synthesis method that may greatly facilitate large-scale fabrication of high-purity spiro-based ionic liquid salt under ambient conditions.

As will be shown in the examples below, while either isopropanol or acetonitrile may be used in Step I, using isopropanol (instead of acetonitrile) in Step I may increase the final product yield. Moreover, the use of isopropanol (instead of acetonitrile) and ethanol in the two-step process may reduce the entire cost of synthesizing this ionic liquid salt at a large commercial scale.

### C. EXAMPLES

### Example 1: Synthesis of Spiro-1,1′ Bipyrrolidinium Bromide (SBPBr) with 40% Yield

Approximately, 50.0 grams (g) (that is, 0.23 mole (mol)) of 1,4-dibromobutane and 34.9 g (0.23 mol) of potassium carbonate are mixed in 200 milliliter (mL) acetonitrile. 15.0 g (0.21 mol) of pyrrolidine is added dropwise to the solution in 30 minutes. The solution is mixed and heated at 340 K for 6 h. Then, the potassium bromide is filtered, and acetonitrile is evaporated with rotary at 350 K. The powder is washed with acetone and dried in the vacuum oven for 12 h.

A sample is identified by ¹H NMR (500 MHz, CD₃OD): δ=2.23 (t, 8H), δ=3.63 (d, 8H).

The reaction in this example is as follows:

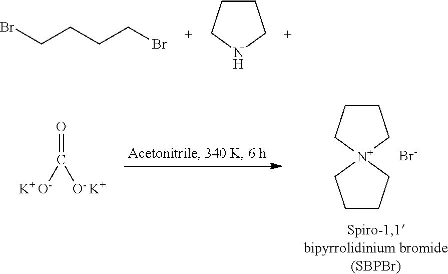

The yield of SBPBr is about 40%.

### Example 2: Synthesis of Spiro-1,1′ Bipyrrolidinium Bromide (SBPBr) with 46% Yield

Approximately, 50.0 g (0.23 mol) of 1,4-dibromobutane and 34.9 g (0.23 mol) of potassium carbonate are mixed in 200 mL acetonitrile. 15.0 g (0.21 mol) of pyrrolidine is added dropwise to the solution in 30 minutes. The solution is mixed and heated at 340 K for 12 hours. Then, the potassium bromide is filtered, and acetonitrile is evaporated with rotary at 350 K. The powder is washed with acetone and dried in the vacuum oven for 12 hours.

A sample is identified by ¹H NMR (500 MHz, CD₃OD): δ=2.23 (t, 8H), δ=3.63 (d, 8H).

The reaction in this example is as follows:

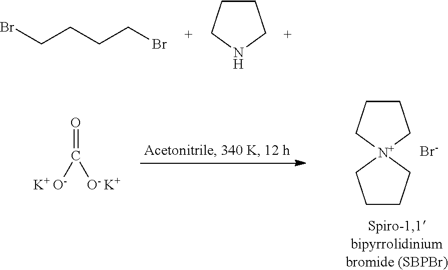

The yield of SBPBr is about 46%.

### Example 3: Synthesis of Spiro-1,1′ Bipyrrolidinium Bromide (SBPBr) with 75% Yield

Approximately, 50.0 g (0.23 mol) of 1,4-dibromobutane and 34.9 g (0.23 mol) of potassium carbonate are mixed in 200 mL acetonitrile. 15.0 g (0.21 mol) of pyrrolidine is added dropwise to the solution in 30 minutes. The solution is mixed and heated at 340 K for 18 hours. Then, the potassium bromide is filtered, and acetonitrile is evaporated with rotary at 350 K. The powder is washed with acetone and dried in the vacuum oven for 12 hours.

A sample is identified by ¹H NMR (500 MHz, CD₃OD): δ=2.23 (t, 8H), δ=3.63 (d, 8H).

The reaction in this example is as follows:

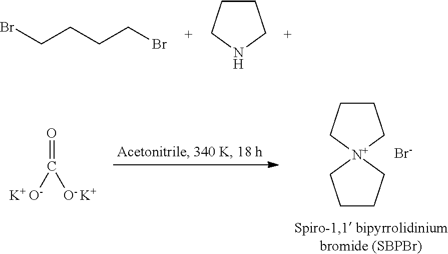

The yield of SBPBr is about 75%.

### Example 4: Synthesis of Spiro-1,1′ Bipyrrolidinium Bromide (SBPBr) with 45% Yield

Approximately, 50.0 g (0.23 mol) of 1,4-dibromobutane and 34.9 g (0.23 mol) of potassium carbonate are mixed in 200 mL acetonitrile. 15.0 g (0.21 mol) of pyrrolidine is added dropwise to the solution in 30 minutes. The solution is mixed and heated at 340 K for 24 hours. Then, the potassium bromide is filtered, and acetonitrile is evaporated with rotary at 350 K. The powder is washed with acetone and dried in the vacuum oven for 12 hours.

A sample is identified by ¹H NMR (500 MHz, CD₃OD): δ=2.23 (t, 8H), δ=3.63 (d, 8H).

The reaction in this example is as follows:

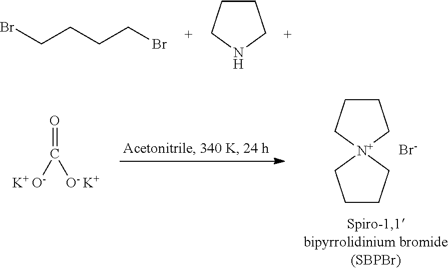

The yield of SBPBr is about 45%.

### Example 5: Synthesis of Spiro-1,1′ Bipyrrolidinium Bromide (SBPBr) with 96% Yield

Approximately, 50.0 g (0.23 mol) of 1,4-dibromobutane and 34.9 g (0.23 mol) of potassium carbonate are mixed in 200 mL isopropanol. 15.0 g (0.21 mol) of pyrrolidine is added dropwise to the solution in 30 minutes. The solution is mixed and heated at 340 K for 18 hours. Then, the potassium bromide is filtered, and isopropanol is evaporated with rotary at 350 K. The powder is washed with acetone and dried in the vacuum oven for 12 hours.

A sample is identified by ¹H NMR (500 MHz, CD₃OD): δ=2.23 (t, 8H), δ=3.63 (d, 8H).

The reaction in this example is as follows:

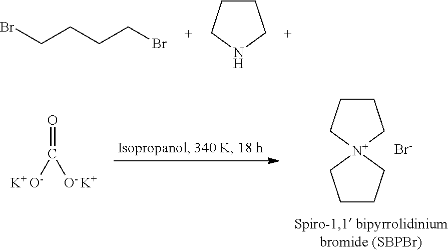

The yield of SBPBr is about 96%.

### Example 6: Synthesis of Spiro-1,1′ Bipyrrolidinium Tetraflouroborate (SBPBF₄) with NaBF₄

40.0 g (0.21 mol) of SBPBr and 30.0 g (0.21 mol) of Sodium tetrafluoroborate are added to 200 mL ethanol and the solution is stirred for 18 hours at room temperature. After filtration, the filtrate solution is evaporated, and the product is recrystallized in ethanol. SBPBF₄ is washed several 10 times with ethanol and is dried in the vacuum oven for 12 hours.

A sample is identified by ¹H NMR (500 MHz, CD₃OD): δ=2.23 (m, 8H), δ=3.55 (m, 8H).

The reaction in this example is as follows:

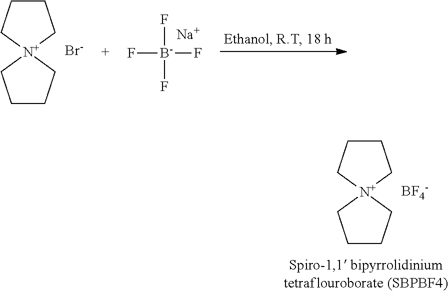

The yield of SBPBF₄ is about 35%.

### Example 7: Synthesis of Spiro-1,1′ Bipyrrolidinium Tetraflouroborate (SBPBF₄) with HBF₄

40.0 g (0.21 mol) of SBPBr and 27.6 g (0.16 mol) of 40% tetraflouroboric acid are added to 200 mL ethanol and the solution is stirred for 18 hours at room temperature. The solution is repeatedly evaporated (greater than or equal to three (3) times) with ethanol to eliminate hydrobromic acid and water. The product is recrystallized in ethanol. SBPBF₄ is washed several times with ethanol and is dried in the vacuum oven for 12 hours.

A sample is identified by ¹H NMR (500 MHz, CD₃OD): δ=2.23 (m, 8H), δ=3.55 (m, 8H).

The reaction in this example is as follows:

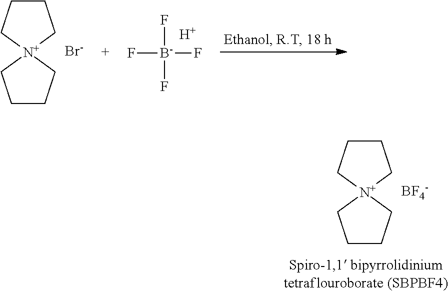

The yield of SBPBF₄ is about 45%.

### Example 8: Synthesis of spiro-1,1′ bipyrrolidinium tetraflouroborate (SBPBF₄) with HBF₄ in base (OH⁻) medium

40.0 g (0.21 mol) of SBPBr and 10.6 g (0.19 mol) of potassium hydroxide are added to 200 mL ethanol and the solution is stirred for 6 hours at room temperature. The solution is filtered and 40% tetraflouroboric acid is added drop by drop to the filtrate until a pH of 5 to 6 is recorded. The solution is evaporated, and the final product is recrystallized in ethanol. The SBPBF₄ is washed several times with ethanol and is dried in the vacuum oven for 12 hours.

The repeated evaporation of hydrobromic acid with ethanol is circumvented by the use of a basic medium for synthesis.

A sample is identified by ¹H NMR (500 MHz, CD₃OD): δ=2.23 (m, 8H), δ=3.55 (m, 8H).

The reaction in this example is as follows:

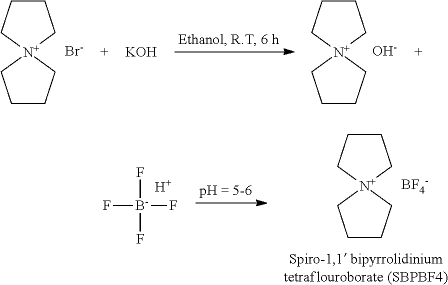

The yield of SBPBF₄ is about 60% with high purity.

The summary of all synthesis steps carried out is shown in Table 1 below.

TABLE 1

| | Reagents | Spiro salt | Reaction time | Results |
|---|---|---|---|---|
| 1. | 0.21 mol Pyrrolidine 0.23 mol 1,4-dibromobutane 0.23 mol Potassium carbonate 200 mL Acetonitrile | 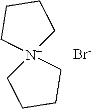 | 6 hours (Step I) | Very low yield |
| 2. | 0.21 mol Pyrrolidine 0.23 mol 1,4-dibromobutane 0.23 mol Potassium carbonate 200 mL Acetonitrile | 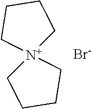 | 12 hours (Step I) | Low yield |
| 3. | 0.21 mol Pyrrolidine 0.23 mol 1,4-dibromobutane 0.23 mol Potassium carbonate 200 mL Acetonitrile | 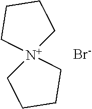 | 18 hours (Step I) | Good yield |
| 4. | 0.21 mol Pyrrolidine 0.23 mol 1,4-dibromobutane 0.23 mol Potassium carbonate 200 mL Acetonitrile | 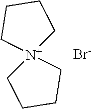 | 24 hours (Step I) | Low yield |
| 5. | 0.21 mol Pyrrolidine 0.23 mol 1,4-dibromobutane 0.23 mol Potassium carbonate 200 mL Isopropanol | 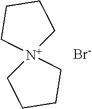 | 18 hours (Step I) | Excellent overall yield |
| 6. | 0.21 mol SBPBr 0.21 mol Sodium tetrafluoroborate 200 mL Ethanol | 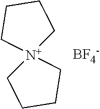 | 18 hours (Step II) | Low yield High purity |
| 7. | 0.21 mol SBPBr 0.16 mol Tetraflouroboric acid 200 mL Ethanol | 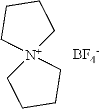 | 18 hours (Step II) | Good yield Low purity |
| 8. | 0.21 mol SBPBr 0.19 mol potassium hydroxide 0.21 mol Tetraflouroboric acid 200 mL Ethanol | 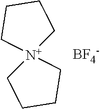 | 18 hours (Step II) | Excellent overall yield High purity |

### D. APPLICATION OF THE SPIRO-BASED IONIC LIQUID ELECTROLYTE IN A LOW TEMPERATURE SUPERCAPACITOR

Activated carbon electrodes coated on etched aluminum isolated from each other by a surfactant-coated polypropylene separator is assembled in a pouch-type packaging using the synthesized electrolytes as described in Examples 1 to 8. Aluminum and Nickel tabs are ultrasonically welded as the positive and negative terminals respectively to prevent excessive degradation during operation (see Reference [3]).

In some embodiments, a free-standing solid-state gel electrolyte may be fabricated by encapsulation of the ionic liquid electrolyte in a gel. The free-standing solid-state gel electrolyte may also be used as the separator layer 108 of a supercapacitor 100 which may be bendable and stretchable supercapacitor.

As those skilled in the art will appreciate, the supercapacitor fabricated as described above is stable and comprises commercial-grade porous activated carbon electrodes operated with the above-described low-temperature ionic liquid electrolyte in much lesser concentrations as compared to commercial electrolytes. More specifically, the precise control of the ion dynamics within the electrolyte may produce efficient charge transport and storage properties even at ultra-low temperatures such as −60° C. In some embodiments, the ionic liquid electrolyte has a low concentration of about 0.1 M.

Self-discharge tests show that the supercapacitor fabricated as described above has superior device-voltage retention (low voltage leakage) at room temperature. The complete capacitance recovery from the supercapacitor fabricated using electrolytes described above is tested at ultra low temperature. Test results show that the supercapacitor fabricated as described above provides improved self-discharge characteristic even after extended ultra-low temperature operation with ionic liquid electrolyte of.

### E. TESTING OF THE SUPERCAPACITOR

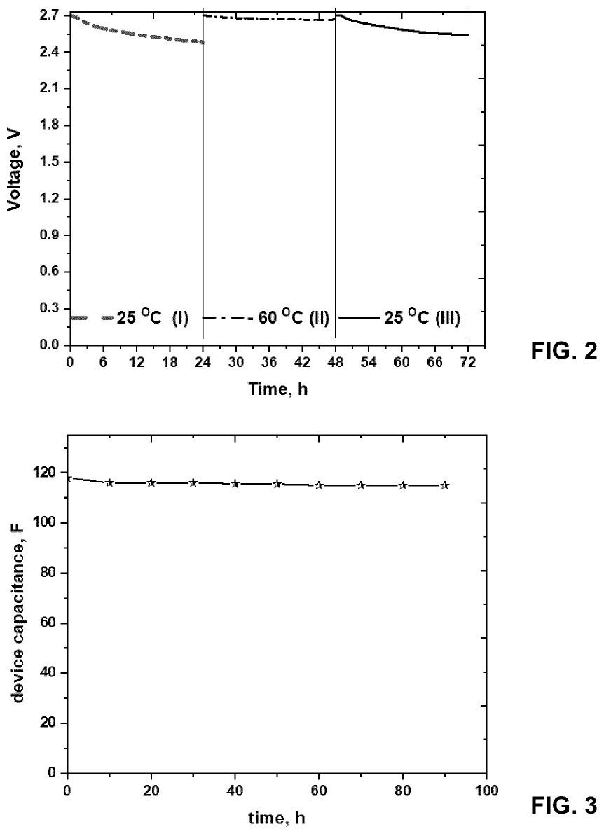

Initial degassing of the supercapacitors is performed before final vacuum sealing after initial cycling. The 100 farad (F) rated supercapacitors are placed in an environmental chamber with programmable temperature test conditions. The device performance over a temperature ranging from 25° C. to −60° C. and −60° C. to 25° C. is tested. In addition, leakage tests are performed at a fixed temperature of −60° C. for up to 24 hours and are compared with normal leakage tests at room temperature. The voltage holding test which is a better form of testing device stability (see Reference [4]) is also performed after the low-temperature tests continuously for up to 90 hours at the maximum 2.7 Volts (V) operating voltage. The results are shown in the Table 2 and FIGS. 2 and 3.

TABLE 2

| Temperature | Capacitance |
|---|---|
| (° C.) | (F) |
| 25 | 120 |
| 0 | 105 |
| −25 | 90 |
| −50 | 60 |
| −60 | 45 |
| 25 | 119 |

### F. REFERENCES

[1] Zhou, Hm., Sun, Wj. & Li, J. Preparation of spiro-type quaternary ammonium salt via economical and efficient synthetic route as electrolyte for electric double-layer capacitor. J. Cent. South Univ. 22, (2015) 2435-2439.
[2] Chiba, Kazumi, Tsukasa Ueda, and Hideo Yamamoto. “Performance of electrolyte composed of spiro-type quaternary ammonium salt and electric double-layer capacitor using it.” Electrochemistry 75.8 (2007): 664-667.
[3] Liu, Yinghui, et al. “Understanding ageing mechanisms of porous carbons in non-aqueous electrolytes for supercapacitors applications.” Journal of Power Sources 434 (2019): 226734.
[4] Weingarth, D., A. Foelske-Schmitz, and R. Kötz. “Cycle versus voltage hold—Which is the better stability test for electrochemical double layer capacitors?.” Journal of Power Sources 225 (2013): 84-88.

Although embodiments have been described above with reference to the accompanying drawings, those of skill in the art will appreciate that variations and modifications may be made without departing from the scope thereof as defined by the appended claims.

1. An electrochemical energy-storage apparatus comprising:
an ionic liquid electrolyte comprising an ionic liquid salt with a cation of:

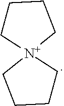

2. The ionic liquid electrolyte of claim 1, wherein the ionic liquid electrolyte is a spiro-based ionic liquid electrolyte.

3. The ionic liquid electrolyte of claim 1, wherein the ionic liquid electrolyte is spiro-1,1′ bipyrrolidinium bromide (SBPBr):

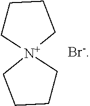

4. An electrochemical energy-storage apparatus comprising:
an ionic liquid electrolyte comprising an ionic liquid salt with an anion of:

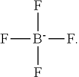

5. The ionic liquid electrolyte of claim 4, wherein the ionic liquid electrolyte is a spiro-based ionic liquid electrolyte.

6. The ionic liquid electrolyte of claim 4, wherein the ionic liquid electrolyte is spiro-1,1′ bipyrrolidinium tetraflouroborate (SBPBF₄):

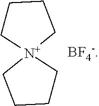

7. A method for fabricating an ionic liquid electrolyte, the method comprising:
synthesizing an intermediate spiro-based product; and
applying an ionic exchange process to the intermediate spiro-based product to obtain the ionic liquid electrolyte.

8. The method of claim 7, wherein said synthesizing the intermediate spiro-based product comprises:
adding a plurality of precursors are added to an organic solvent to obtain a mixture, and stirring the mixture for a period of time at a temperature of 340 Kelvin (K) to obtain the intermediate spiro-based product; and
purifying the intermediate spiro-based product;
wherein the plurality of precursors comprises alkylation of a cyclic amine and a dihaloalkane.

9. The method of claim 7, wherein the organic solvent comprises isopropanol or acetonitrile.

10. The method of claim 8, wherein the period of time is about 6 hours (h), 12 h, 18 h, or 24 h.

11. The method of claim 8, wherein said purifying the intermediate spiro-based product comprises:
washing the intermediate spiro-based product with acetone.

12. The method of claim 8, wherein said applying the ionic exchange process to the intermediate spiro-based product to obtain the ionic liquid electrolyte comprises:
reacting the intermediate spiro-based product with hydrofluoroboric acid or an alkali-metal tetrafluoroborate salt in ethanol.
